(19) United States
(12) Patent Application Publication
(10) Pub. No.: US 2013/0030407 A1
(43) Pub. Date: Jan. 31, 2013

(54) Fluidic components, systems, and methods for use in ophthalmic surgeries

(71) Applicant: John M. Bourne, Tustin, CA (US)

(72) Inventor: John M. Bourne, Tustin, CA (US)

(21) Appl. No.: 13/465,586

(22) Filed: May 7, 2012

Related U.S. Application Data
(60) Provisional application No. 61/512,954, filed on Jul. 29, 2011.

(51) Int. Cl. A61M 5/36; A61F 9/007; A61M 5/38

(52) U.S. Cl. 604/500; 604/122; 604/126

(57) ABSTRACT

Fluidic components, systems, and methods for use in ophthalmic surgeries are disclosed. In some embodiments, a fluidics cassette for use with an ophthalmic surgical console system is provided. The fluidics cassette includes a housing, a fluid passageway extending through the housing, a membrane positioned in fluid communication with the fluid passageway such that a fluid passing through the fluid passageway contacts the membrane, and an opening extending at least partially through the housing such that the opening is in communication with the membrane and is configured to be coupled to a vacuum source. When the vacuum source is coupled to the opening, fluid passing through the passageway is subjected to the vacuum generated by the vacuum source through the membrane to remove dissolved gasses from the fluid.

This application claims the priority of U.S. Provisional Application No. 61/512,954 filed Jul. 29, 2011.

### BACKGROUND

The present disclosure relates generally to ophthalmic surgical systems, and, more particularly, to fluidic systems used for intraocular surgeries that remove gasses from solutions introduced into the eye during such surgeries.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

Often, cataractous lenses are removed by a surgical technique called phacoemulsification. During this procedure, a thin phacoemulsification cutting tip is inserted into the diseased lens and vibrated ultrasonically. The vibrating cutting tip liquefies or emulsifies the lens so that the lens may be aspirated out of the eye. An irrigation fluid may be utilized to assist in removal of the emulsified lens. The diseased lens, once removed, is replaced by an artificial lens.

Another cataract removal technique has been developed that involves the injection of hot (approximately 45° C. to 105° C.) solution to liquefy or gellate the hard lens nucleus, thereby making it possible to aspirate the liquefied lens from the eye. Aspiration is conducted with the injection of the heated solution and the injection of a relatively cool solution, thereby quickly cooling and removing the heated solution. An exemplary technique of this nature is more fully described in U.S. Pat. No. 5,616,120 (Andrew, et al.), which is hereby incorporated by reference in its entirety. A commercially available device that practices the liquefaction method is the AQUALASE® hand piece, part of the INFINITI® Vision System available from Alcon Laboratories, Inc., Fort Worth, Tex.

The introduction of air bubbles into the eye is very common during both ultrasonic phacoemulsification and non-ultrasonic removal of lens material by irrigation and aspiration. In that regard, heating of a solution and/or a reduction in pressure can cause the release of dissolved gasses that exist in the solution. The release of the gasses can become a nuisance to the medical personnel performing the procedure as the resulting bubbles interfere with the field of view. As a result, the bubbles must be manually aspirated or pushed off to a periphery of the field of view (e.g., using a viscoelastic).

While various attempts have been made in the past to limit or prevent the production of bubbles during ophthalmic surgeries, better techniques are needed. Particularly, as addressed by the present disclosure, a need continues to exist for ophthalmic surgical systems that include fluidics components that remove both bubbles and dissolved gasses that result in bubbles from solutions before application of the solutions to the eye.

### SUMMARY

The present disclosure provides ophthalmic surgical systems and associated fluidics components and methods for use in ophthalmic surgery.

In one embodiment, a fluidics cassette for use with an ophthalmic surgical console system is provided. The fluidics cassette includes a housing, a fluid passageway extending through the housing, a membrane positioned in fluid communication with the fluid passageway such that a fluid passing through the fluid passageway contacts the membrane, and an opening extending at least partially through the housing. The opening is in communication with the membrane and configured to be coupled to a vacuum source such that, when the vacuum source is coupled to the opening, fluid passing through the passageway is subjected to the vacuum generated by the vacuum source through the membrane. In some instances, the membrane is configured to allow passage of gasses through the membrane and prevent passage of the fluid through the membrane. In that regard, the membrane is hydrophobic in some embodiments.

In some instances, the membrane extends generally parallel to the fluid passageway. In other instances, the membrane extends generally perpendicular to the fluid passageway. In yet other instances, the membrane extends at an oblique angle to the fluid passageway. In some embodiments, the cassette includes multiple membranes positioned in fluid communication with the fluid passageway. In that regard, each of the multiple membranes may extend in the same and/or a different orientation (i.e., parallel, perpendicular, or oblique) relative to the fluid passageway as other of the multiple membranes.

In some embodiments, the fluid passageway is defined by an inlet, an outlet, and a lumen extending between the inlet and the outlet. Further, in some instances the inlet is configured to receive the fluid from a fluid source and the outlet is configured to output the fluid to a surgical instrument after dissolved gasses have been extracted from the fluid through the membrane.

In another embodiment, an ophthalmic surgical system is provided. The ophthalmic surgical system includes a fluid source, a vacuum source, a fluid passageway in fluid communication with the fluid source such that a fluid from the fluid source passes through the fluid passageway, a membrane positioned in communication with the fluid passageway such that the fluid passing through the fluid passageway contacts the membrane, and a vacuum passageway in communication with the vacuum source and the membrane such that a vacuum generated by the vacuum source is applied through the membrane to the fluid passing through the fluid passageway. In some instances, the membrane is configured to allow passage of gasses through the membrane and prevent passage of the fluid through the membrane. In that regard, the membrane is hydrophobic in some embodiments.

In some instances, the membrane of the system extends generally parallel to the fluid passageway. In other instances, the membrane of the system extends generally perpendicular to the fluid passageway. In yet other instances, the membrane of the system extends at an oblique angle to the fluid passageway. In some embodiments, the system includes multiple membranes positioned in fluid communication with the fluid passageway. In that regard, each of the multiple membranes may extend in the same and/or a different orientation (i.e., parallel, perpendicular, or oblique) relative to the fluid passageway as other of the multiple membranes. In some instances, at least portions of each of the fluid passageway, the membrane, and the vacuum passageway of the system are positioned within a fluidics cassette.

In another embodiment, an ophthalmic surgical method is provided. The surgical method includes introducing a fluid into a fluid passageway, wherein a membrane is positioned in communication with the fluid passageway such that the fluid passing through the fluid passageway contacts the membrane. The method also includes applying a vacuum to the fluid through the membrane such that dissolved gasses within the fluid are removed through the membrane and introducing the fluid into the eye after removal of the dissolved gasses through the membrane. In some instances, the fluid is an electrolyte solution. Further, in some embodiments the membrane is hydrophobic such that the fluid does not pass through the membrane.

Other aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a front view of an ophthalmic surgical console system according to one embodiment of the present disclosure.

FIG. 2 is a top view of a fluidics cassette of the ophthalmic surgical console system of FIG. 1 according to one embodiment of the present disclosure.

FIG. 3 is a bottom view of the fluidics cassette of FIG. 2.

FIG. 4 is a diagrammatic schematic cross-sectional side view of a filtering arrangement according to one embodiment of the present disclosure.

FIG. 5 is a diagrammatic schematic cross-sectional, close-up side view of a portion of the filtering arrangement of FIG. 4.

FIG. 6 is a diagrammatic schematic cross-sectional side view of a filtering arrangement according to another embodiment of the present disclosure.

FIG. 7 is a diagrammatic schematic cross-sectional side view of a filtering arrangement according to another embodiment of the present disclosure.

FIG. 8 is a diagrammatic schematic cross-sectional side view of a filtering arrangement according to another embodiment of the present disclosure.

FIG. 9 is a diagrammatic schematic cross-sectional side view of a filtering arrangement according to another embodiment of the present disclosure.

FIG. 10 is a diagrammatic schematic cross-sectional side view of a filtering arrangement according to another embodiment of the present disclosure.

FIG. 11 is a diagrammatic schematic cross-sectional side view of a filtering arrangement according to another embodiment of the present disclosure.

FIG. 12 is a diagrammatic schematic cross-sectional side view of a filtering arrangement according to another embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described individually.

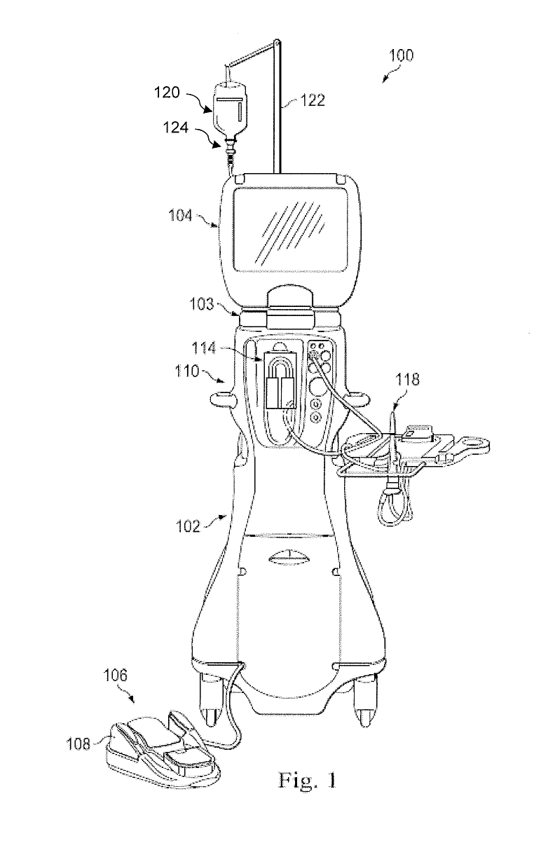

Referring to FIG. 1, shown therein is an ophthalmic surgical console system, generally designated 100, according to an exemplary embodiment of the present disclosure. The console system 100 includes a base housing 102 with a computer unit 103 and an associated display screen 104. The system 100 also includes a number of subsystems that are used to perform ophthalmic procedures. For example, the system 100 includes a foot pedal subsystem 106—including a foot pedal 108—that is utilized to control operation of various aspects of the system. The system 100 also includes a fluidics subsystem 110. Generally, the fluidics system 110 includes a vacuum source (not shown) and fluidics cassette 114. The vacuum source and the fluidics cassette are coupled to a hand piece 118 via tubing. In that regard, the vacuum source is coupled to the hand piece 118 to allow aspiration of fluid from the eye, while the cassette 114 is coupled to the hand piece to allow the application of irrigation fluid to the eye. In some instances, the hand piece 118 is an ultrasonic hand piece. In some particular instances, the hand piece 118 includes features similar to those of the OZil® Torsional hand piece available from Alcon Laboratories, Inc., Fort Worth, Tex. In some instances, the hand piece 118 is a liquefaction hand piece. In some particular instances, the hand piece 118 includes features similar to those of the AQUALASE® hand piece available from Alcon Laboratories, Inc., Fort Worth, Tex.

Generally, the fluidics cassette 114 is coupled to a fluid source. In the illustrated embodiment, the fluid source is a container 120 (e.g., a bottle, bag, or other fluid holding container) that is attached to an IV pole 122. The container 120 holds a fluid for use by the system 100. In some instances, the fluid is an irrigating solution. In that regard, for some procedures the irrigating solution is a saline solution or a balanced salt solution. In some particular instances, the fluid is BSS PLUS® available from Alcon Laboratories, Inc., Fort Worth, Tex. Tubing extends between the container 120 and the fluidics cassette 114 to fluidly couple the fluid to the cassette. In some instances, a drip chamber 124 or other fluid flow-limiting device is positioned between the container 120 and the cassette 114.

The ophthalmic surgical console system 100 is provided by way of example and embodiments of the present disclosure can be implemented with a variety of ophthalmic surgical systems. Examples of ophthalmic surgical systems in which embodiments of the present disclosure can be implemented include, for example, the Infiniti® Vision System surgical system available from Alcon Laboratories Inc. of Fort Worth, Tex. Persons skilled in the art will appreciate that the embodiments described below can be utilized with other types of surgical equipment including, but not limited to, any surgical systems where it is desirable to introduce a fluid with as few bubbles as possible, including no bubbles. However, for purposes of explanation, not limitation, the remainder of this specification describes embodiments related to ophthalmic systems and associated procedures.

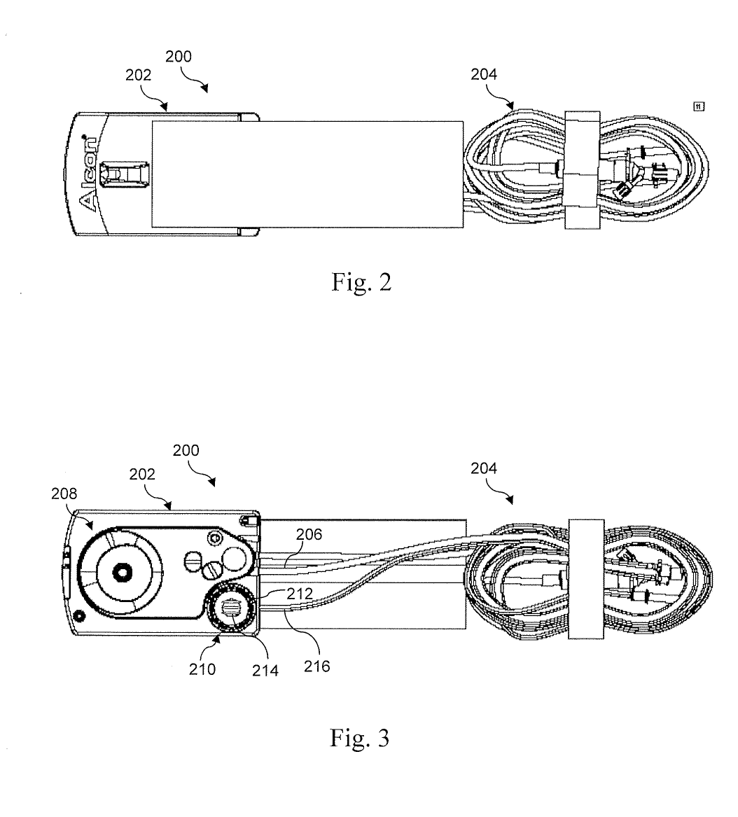

Referring now to FIGS. 2 and 3, shown therein is a fluidics cassette 200 according to an exemplary embodiment of the present disclosure. As shown, the fluidics cassette 200 includes a housing 202. Generally, the housing 202 contains the working components of the cassette 100 and, in some embodiments, is configured to be received by an ophthalmic surgical console. The cassette 100 is shown coupled to tubing 204. As discussed below, the tubing 204 provides fluidly couples the cassette 100 to other components of an ophthalmic surgical system, such as a fluid source and a hand piece.

Referring more specifically to FIG. 3, an inlet tubing 206 is provided to allow the introduction of fluid to the cassette. Generally, the inlet tubing 206 is coupled to a fluid source, such as container 120 of FIG. 1. The inlet tubing 206 is fluidly coupled to a mechanism 208 of the cassette 100. Depending on the type of procedures the cassette is designed to be utilized with, the mechanism 208 can take the form of a pump configured to pump the fluid to the hand piece, a heating mechanism configured to heat the fluid, a combination pump and heating mechanism, and/or other arrangement configured to provides functionalities related to an ophthalmic procedure.

A filtering mechanism 210 is fluidly coupled to the output of the mechanism 208. The filtering mechanism 210 is configured to reduce the number of bubbles introduced into the eye by the hand piece. In that regard, the filtering mechanism 210 is arranged to remove dissolved gasses and un-dissolved gasses (i.e., bubbles) from the fluid as the fluid passes through the filtering mechanism. Accordingly, in some instances the filtering mechanism 210 includes at least one membrane 212 coupled to a vacuum source 214. In that regard, the at least one membrane 212 is configured such that gasses are able to pass through the membrane, while the fluid is forced to go around the membrane. Accordingly, when vacuum is applied to the at least one membrane 212 as fluid is passing through the filtering mechanism 210, the gasses dissolved in the fluid are extracted through the membrane, along with any bubbles, such that the fluid that passes through the filtering mechanism and on towards the hand piece through outlet tubing 216 will not generate bubbles when introduced to the eye. By eliminating the production of bubbles, the need to manually aspirate the bubbles or use a viscoelastic to move the bubbles out of the field of view is also eliminated. Further, removing the dissolved gasses from the fluid also serves to reduce the magnitude of occlusion surges and/or delay the onset of cavitation development.

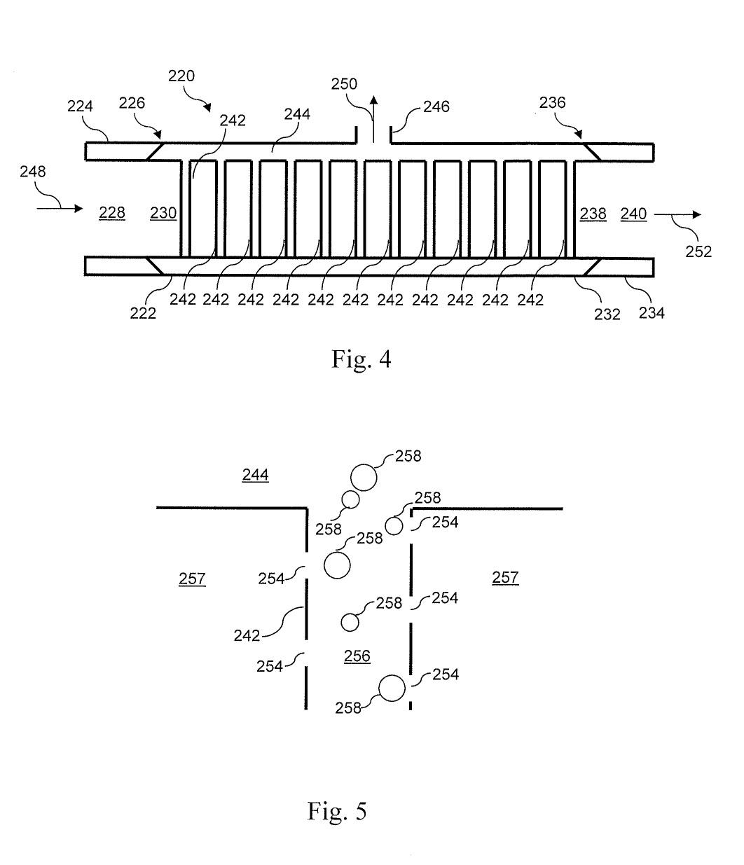

Referring now to FIGS. 4-12, shown therein are various implementations of filtering arrangements in accordance with the present disclosure. In that regard, the exemplary filtering arrangements illustrated in FIGS. 4-12 are suitable for implementation as the filtering mechanism 210 in cassette 200 described above with respect to FIGS. 2 and 3.

Referring to FIG. 4, shown therein is a filtering mechanism 220 according to an exemplary embodiment of the present disclosure. In that regard, a proximal portion 222 of the filtering mechanism 220 is coupled to a fluid pathway defining structure 224 (such as a tube or other structure intended to carry fluid) at an interface 226. In that regard, the interface 226 between the proximal portion 222 of the filtering mechanism 220 and the fluid pathway defining structure 224 fluidly couples a lumen 228 of the structure 224 to a lumen 230 of the filtering mechanism 220. Accordingly, the interface 226 can take any suitable form for fluidly coupling the lumens 228 and 230, including threaded, press-fit, luer, and/or any other suitable connection type. Similarly, a distal portion 232 of the filtering mechanism 220 is coupled to a fluid pathway defining structure 234 (such as a tube or other structure intended to carry fluid) at an interface 236. Again, the interface 236 between the distal portion 232 of the filtering mechanism 220 and the fluid pathway defining structure 234 fluidly couples a lumen 238 of the filtering mechanism 220 to a lumen 240 of the structure 234. In that regard, the lumen 238 adjacent the distal portion 232 of the filtering mechanism 220 is fluidly coupled to the lumen 230 adjacent the proximal portion 222 of the filtering mechanism. In some instances, the lumens 230 and 238 are portions of a single continuous lumen.

The lumens 228, 230, 238, and 240 generally define a fluid passageway along which a fluid introduced will travel. In the illustrated embodiment, a plurality of membranes 242 extend across the fluid passageway between the proximal and distal lumens portions 230, 238 of the filtering mechanism. Accordingly, as the fluid travels from the proximal lumen portion 230 to the distal lumen portion 238 the fluid will travel over and/or around the plurality of membranes 242. As shown, each of the plurality of membranes 242 extends generally perpendicular to the longitudinal axis of the fluid passageway. In some embodiments, each of the plurality of membranes 242 is a tubular structure. Further, while the plurality of membranes 242 are shown as having an equal spacing from one another, it is understood that the plurality of membranes 242 can be arranged in any suitable way to encourage fluid passing through the filtering mechanism 220 to pass over and around the membranes 242, including symmetrical and non-symmetrical spacings and layouts. Further, it is understood that while a single membrane 242 is illustrated at each point along the longitudinal axis of the fluid passageway in the cross-sectional view of FIG. 4, in some embodiments there are one or more additional membranes adjacent to the illustrated membrane at that point along the longitudinal axis of the fluid passageway. Finally, it is understood that while the filtering mechanism 220 is illustrated as having twelve individual membranes 242, this is simply for clarification purposes to illustrate the general concept of the filtering mechanism 220. It is understood that in some instances, the number of membranes will be greater than twelve, including greater than 100, greater than 1,000, greater than 10,000, and greater than 100,000 in some embodiments.

The plurality of membranes 242 are in communication with a chamber 244. In that regard, a port 246 of the filtering mechanism is configured to be coupled to a vacuum source. When the vacuum source is actuated while coupled to the port 246, chamber 244 becomes a vacuum chamber. When the vacuum is applied to the chamber 244 it is also applied to the plurality of membranes 242 that are in communication with the chamber. Each of the plurality of membranes 242 is configured such that gasses are able to pass through the membrane, while the fluid is forced to go around the membrane. In that regard, the membranes 242 are hydrophobic membranes in some instances. In some specific instances, the membranes 242 are formed of polyolefin, polyethylene, epoxy, and/or combinations thereof. In some instances, the hydrophobic membranes include features similar to those found in the SuperPhobic® membrane contactors available from Membrana—Charlotte, A Division of Celgard, LLC. In that regard, the membranes 242 are configured to extract gasses, both dissolved and un-dissolved, from the fluid as the fluid flows over and around the membranes.

In use, a vacuum is applied to the filtering mechanism 220 via port 246. As fluid enters the proximal end of the filtering mechanism 220, as indicated by arrow 248, the fluid will begin passing over and/or around the plurality of membranes 242. As the fluid passes over and/or around the plurality of membranes 242, the vacuum being applied to the membranes 242 from chamber 242 will cause the gasses present within the fluid to be extracted. In that regard, arrow 250 illustrates the flow of the extracted gasses out of port 246. As a result of the gasses being extracted, the fluid that is emitted from the distal end of the filtering mechanism 220 and into lumen 240, as indicated by arrow 252, will generate fewer, if any, bubbles when subsequently introduced into the eye.

Referring to FIG. 5, shown therein is a cross-sectional, close-up side view of a portion of the filtering mechanism 220 of FIG. 4 that pictorially illustrates the functionality of a membrane 242. In that regard, the membrane 242 is a micro-porous structure in some instance. In such instances, the membrane 242 includes a plurality of openings 254 that allow the passage of gasses therethrough to a lumen 256. However, the openings 254 do not allow the passage of the fluid therethrough. Accordingly, only gasses present within the fluid are able to pass through the membrane and be extracted. In that regard, the extracted gasses are illustrated in FIG. 5 as gas bubbles 258. Due to the vacuum source applied to chamber 244, the extracted gasses 258 are drawn upwards through the lumen 256 of the membrane 242 towards chamber 244.

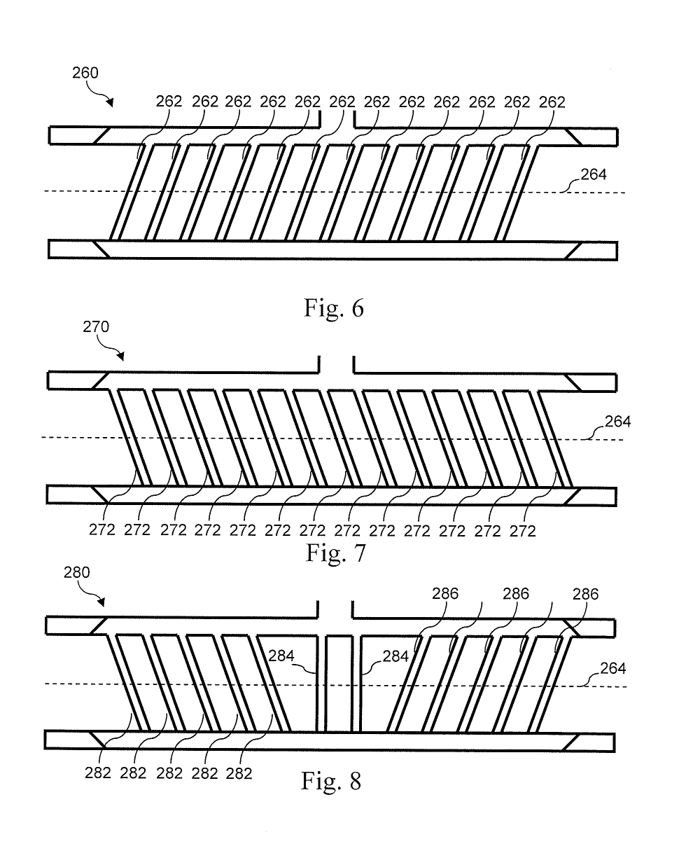

As noted above, it is understood that the membranes of the present disclosure can be arranged in a variety of ways. FIGS. 6-12 illustrate exemplary alternative embodiments of membrane arrangements. Referring to FIG. 6, shown therein is a filtering mechanism 260 that is similar to filtering mechanism 220 described above in many respects. However, the filtering mechanism 260 includes a plurality of membranes 262 that extend at an oblique angle with respect to a longitudinal axis 264 of the fluid passageway. More specifically, each of the plurality of membranes 262 is arranged such that a lower portion of the membrane (as viewed in FIG. 6) is closer to a proximal end of the filtering mechanism 260 than an upper portion of the membrane. Generally, the membranes 262 extend at an oblique angle between about 1 degree and about 179 degrees relative to the longitudinal axis. In some embodiments, the oblique angle is between about 60 degrees and about 120 degrees. Further, while the illustrated embodiment shows all of the membranes 262 extending at the same oblique angle relative to the longitudinal axis, in other embodiments at least one of the plurality of membranes 262 extends at an oblique angle that is different than another of the plurality of membranes 262.

Referring to FIG. 7, shown therein is a filtering mechanism 270 that is similar to filtering mechanisms 220 and 260 described above in many respects. However, the filtering mechanism 270 includes a plurality of membranes 272 that extend at an oblique angle with respect to a longitudinal axis 264 of the fluid passageway. More specifically, each of the plurality of membranes 272 is arranged such that an upper portion of the membrane (as viewed in FIG. 7) is closer to a proximal end of the filtering mechanism 270 than a lower portion of the membrane. Generally, the membranes 272 extend at an oblique angle between about 1 degree and about 179 degrees relative to the longitudinal axis. In some embodiments, the oblique angle is between about 60 degrees and about 120 degrees. Further, while the illustrated embodiment shows all of the membranes 272 extending at the same oblique angle relative to the longitudinal axis, in other embodiments at least one of the plurality of membranes 272 extends at an oblique angle that is different than another of the plurality of membranes 272.

Referring to FIG. 8, shown therein is a filtering mechanism 280 that is similar to filtering mechanisms 220, 260, and 270 described above in many respects. However, the filtering mechanism 280 includes a plurality of membranes 282 that extend at first oblique angle with respect to a longitudinal axis 264 of the fluid passageway, a plurality of membranes 284 that extend perpendicular to the longitudinal axis, and a plurality of membranes 286 that extend at a second oblique angle with respect to the longitudinal axis. More specifically, each of the plurality of membranes 282 is arranged such that an upper portion of the membrane (as viewed in FIG. 8) is closer to a proximal end of the filtering mechanism 280 than a lower portion of the membrane, while each of the plurality of membranes 286 is arranged such that a lower portion of the membrane (as viewed in FIG. 8) is closer to a proximal end of the filtering mechanism 280 than an upper portion of the membrane.

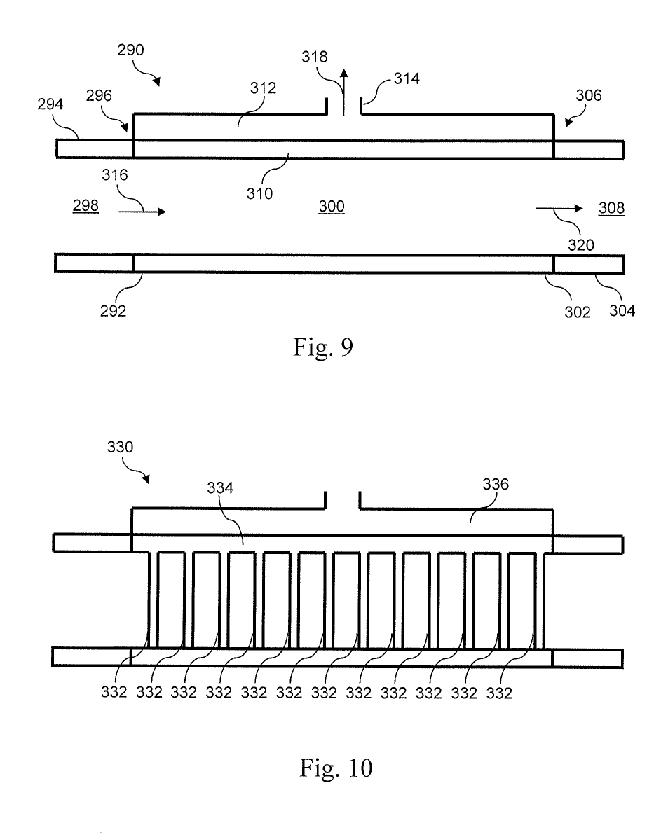

Referring now to FIG. 9, shown therein is a filtering mechanism 290 according to another embodiment of the present disclosure. A proximal portion 292 of the filtering mechanism 290 is coupled to a fluid pathway defining structure 294 (such as a tube or other structure intended to carry fluid) at an interface 296. In that regard, the interface 296 between the proximal portion 292 of the filtering mechanism 290 and the fluid pathway defining structure 294 fluidly couples a lumen 298 of the structure 294 to a lumen 300 of the filtering mechanism 290. The interface 296 can take any suitable form for fluidly coupling the lumens 298 and 300, including threaded, press-fit, luer, and/or any other suitable connection type. Similarly, a distal portion 302 of the filtering mechanism 290 is coupled to a fluid pathway defining structure 304 (such as a tube or other structure intended to carry fluid) at an interface 306. Again, the interface 306 between the distal portion 302 of the filtering mechanism 290 and the fluid pathway defining structure 304 fluidly couples the lumen 300 of the filtering mechanism 290 to a lumen 308 of the structure 304.

The lumens 298, 300, and 308 generally define a fluid passageway along which a fluid will travel through the filtering arrangement. In the illustrated embodiment, a membrane 310 extends along the length of the fluid passageway between the proximal and distal portions 292 and 302 of the filtering mechanism 290. As shown, the membrane 310 extends generally parallel to the longitudinal axis of the fluid passageway. In some instances, the membrane 310 extends along substantially the entire length of the filtering mechanism 290. In other instances, the membrane 310 extends along only a portion of the length of the filtering mechanism 290. For example, the membrane extends across between about 10 percent and about 90 percent of the length of the filtering mechanism 290, in some instances, and extends across between about 30 percent and about 70 percent of the length of the filtering mechanism in other instances.

As fluid travels through lumen 300 the fluid will travel over the membrane 310. The membrane 310 is in communication with a chamber 312. In that regard, a port 314 of the filtering mechanism 290 is configured to be coupled to a vacuum source. When the vacuum source is actuated while coupled to the port 314, chamber 312 becomes a vacuum chamber. When the vacuum is applied to the chamber 312 it is also applied to the membrane 310 that is in communication with the chamber. In that regard, the membrane 310 is configured such that gasses are able to pass through the membrane, but fluids are not. In that regard, the membrane 310 is hydrophobic membranes in some instances. In some specific instances, the membrane 310 is formed of polyolefin, polyethylene, epoxy, and/or combinations thereof. In some instances, the hydrophobic membrane includes features similar to those found in the SuperPhobic® membrane contactors available from Membrana—Charlotte, A Division of Celgard, LLC. Generally, the membrane 310 is configured to extract gasses, both dissolved and un-dissolved, from the fluid as the fluid flows through the lumen 300 and over the membrane.

In use, a vacuum is applied to the filtering mechanism 290 via port 314. As fluid enters the proximal end of the filtering mechanism 290, as indicated by arrow 316, the fluid will begin passing over the membrane 310. As the fluid passes over the membrane 310, the vacuum being applied to the membrane from chamber 312 causes the gasses present within the fluid to be extracted. In that regard, arrow 318 illustrates the flow of the extracted gasses out of port 314. As a result of the gasses being extracted, the fluid that is emitted from the distal end of the filtering mechanism 290 and into lumen 308, as indicated by arrow 320, will generate fewer, if any, bubbles when subsequently introduced into the eye.

While the membrane 310 is illustrated as being only on one side of the lumen 300 (the upper side as viewed on FIG. 9), in other embodiments the membrane is a tubular structure that is generally concentric with a longitudinal axis of the fluid passageway defined by the lumen 300 such that the membrane substantially surrounds the lumen. Similarly, in some embodiments, the chamber is also concentric with the longitudinal axis of the fluid passageway defined by the lumen 300 such that the chamber substantially surrounds the lumen. In some particular embodiments, the membrane is concentrically positioned around the lumen and the cavity is concentrically positioned around the membrane. Further, it is understood that a plurality of membranes are positioned around the lumen 300 in some instances. In some embodiments, the plurality of membranes are symmetrically spaced about the circumference or perimeter (for non-circular cross-sections) of the lumen 300. In other embodiments, the plurality of membranes are non-symmetrically spaced about the circumference or perimeter of the lumen 300.

Referring to FIG. 10, shown therein is a filtering mechanism 330 that is similar to filtering mechanisms 220 and 290 described above in many respects. In that regard, filtering mechanism 330 provides a combination of the membrane orientations illustrated by filtering mechanisms 220 and 290. More specifically, as shown the filtering mechanism 330 includes a plurality of membranes 332 that extend across the fluid passageway in a direction substantially perpendicular to the longitudinal axis of the passageway. The filtering mechanism 330 also includes at least one membrane 334 extending parallel to the longitudinal axis of the passageway. Both the plurality of membranes 332 extending perpendicular to the passageway and the membrane 334 extending parallel to the passageway are in communication with a chamber 336. Accordingly, when a vacuum is applied to the chamber a vacuum is likewise applied to the membranes 332 and 334 to facilitate the removal of gasses from a fluid traveling along the fluid passageway.

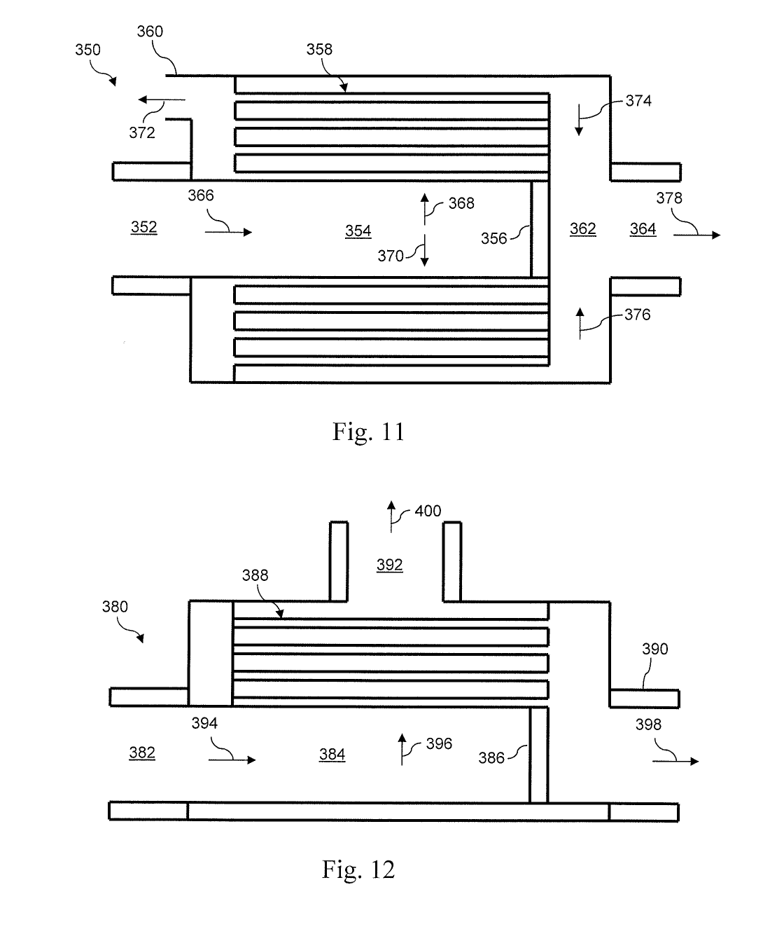

Referring now to FIG. 11, shown therein is a filtering mechanism 350 according to another embodiment of the present disclosure. As shown, a lumen 352 is fluidly coupled to a lumen 354 of the filtering mechanism 350. However, a distal portion of lumen 354 is blocked by a structure 356 and the portions of the lumen 354 proximal of the structure 356 are surrounded by a filter 358. In that regard, the structure 356 blocks the lumen 354 such that fluid must flow through the filter 358 to reach lumen 362 and exit the filtering mechanism 350 to lumen 364.

The filter 358 is comprised of a plurality of membranes. In the illustrated embodiment, the membranes of the filter 358 are shown extending parallel to the longitudinal axis of the lumen 354 and radially surround the lumen. However, the filter 358 may have other membrane structures in other embodiments. The filter 358 is in communication with a port 360 that is configured to be coupled to a vacuum source. When the vacuum source is actuated while coupled to the port 360, a vacuum is applied to the filter 358. In that regard, the filter 358 is configured such that gasses are able to pass through the membranes of the filter, but fluids are not. In that regard, the membranes of the filter 358 are hydrophobic membranes in some instances. In some specific instances, the membranes are formed of polyolefin, polyethylene, epoxy, and/or combinations thereof. In some instances, the membranes include features similar to those found in the SuperPhobic® membrane contactors available from Membrana—Charlotte, A Division of Celgard, LLC.

In use, a vacuum is applied to the filtering mechanism 350 via port 360. As fluid enters the proximal end of the filtering mechanism 350 and travels along lumen 354, as indicated by arrow 366, the fluid will reach structure 356. Structure 356 will cause the fluid to be diverted through the filter 358, as indicated by arrows 368 and 370. As the fluid passes through the filter 358, the fluid will pass over and around the plurality of membranes. The vacuum being applied to the filter 358 causes the gasses present within the fluid to be extracted through the membrane. In that regard, arrow 372 illustrates the flow of the extracted gasses out of port 360. After the gasses have been extracted by the filter 358, the fluid will continue through the filtering mechanism 350 towards lumen 362, as indicted by arrows 374 and 376. Accordingly, the fluid that is emitted from the distal end of the filtering mechanism 350 and into lumen 362, as indicated by arrow 378, will generate fewer, if any, bubbles when subsequently introduced into the eye.

Referring now to FIG. 12, shown therein is a filtering mechanism 380 according to another embodiment of the present disclosure. The filtering mechanism 380 includes some features similar to those of filtering mechanism 350. For example, a lumen 382 is fluidly coupled to a lumen 384 of the filtering mechanism 380 and a distal portion of lumen 384 is blocked by a structure 386. However, a filter 388 is positioned around only a portion of the lumen 384 proximal of the structure 356. As illustrated, the filter 388 is positioned on one side of the lumen 384 (the upper side as viewed in FIG. 12). The structure 386 blocks the lumen 354 such that fluid must flow through the filter 388 to exit the filtering mechanism 350 to lumen 392.

The filter 388 is comprised of a plurality of membranes. In the illustrated embodiment, the membranes of the filter 388 are shown extending parallel to the longitudinal axis of the lumen 384. However, the filter 388 may have other membrane structures in other embodiments. The filter 388 is in communication with a port 390 that is configured to be coupled to a vacuum source. When the vacuum source is actuated while coupled to the port 390, a vacuum is applied to the filter 388. In that regard, the filter 388 is configured such that gasses are able to pass through the membranes of the filter, but fluids are not. In that regard, the membranes of the filter 388 are hydrophobic membranes in some instances. In some specific instances, the membranes are formed of polyolefin, polyethylene, epoxy, and/or combinations thereof. In some instances, the membranes include features similar to those found in the SuperPhobic® membrane contactors available from Membrana—Charlotte, A Division of Celgard, LLC.

In use, a vacuum is applied to the filtering mechanism 380 via port 390. As fluid enters the proximal end of the filtering mechanism 380 and travels along lumen 384, as indicated by arrow 394, the fluid will reach structure 386. Structure 386 causes the fluid to be diverted through the filter 388, as indicated by arrow 396. As the fluid passes through the filter 388, the fluid will pass over and around the plurality of membranes. The vacuum being applied to the filter 388 causes the gasses present within the fluid to be extracted through the membrane. In that regard, arrow 398 illustrates the flow of the extracted gasses out of port 390. After the gasses have been extracted by the filter 388, the fluid will continue through the filtering mechanism 350 an out through lumen 392, as indicted by arrow 400. As a result of the filtering mechanism 380, the fluid that is emitted into lumen 392 will generate fewer, if any, bubbles when subsequently introduced into the eye.

It is understood that any number of additional variations and combinations of membrane structures are contemplated by the present disclosure, but for sake of brevity will not be explicitly shown. However, it should be noted that it is specifically understood that any combination of any portion(s) of any the various arrangements disclosed herein may be combined to form an additional alternative arrangement.

While embodiments of the filtering arrangements of the present disclosure have been described in the context of a fluidics cassette, it is not necessary that the filtering arrangements be positioned within a cassette. Rather, it is understood that the filtering arrangements may be implemented anywhere along the fluid passageway between a fluid source and the output of the hand piece or other surgical instrument where the fluid is dispensed. For example, in some embodiments the filtering arrangement is positioned within and/or adjacent to an output of the fluid source. In such embodiments, the filtering arrangement may be configured to interface with the fluid source, IV pole, drip chamber, or other structure adjacent the fluid source. In other embodiments, the filtering arrangement is positioned within and/or adjacent to a hand piece.

Further, it is understood that in some instances a plurality of filtering arrangements are utilized along the fluid passageway between the fluid source and the output of the hand piece or other surgical instrument where the fluid is dispensed. In that regard, in some embodiments at least one of the plurality of filtering arrangements has a different structure than another of the plurality of filtering arrangements. In other embodiments, all of the plurality of filtering arrangements have the same structure.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure

What is claimed is:

1. A fluidics cassette for use with an ophthalmic surgical console system, comprising:
a housing;
a fluid passageway extending through the housing,
a membrane positioned in fluid communication with the fluid passageway such that a fluid passing through the fluid passageway contacts the membrane;
an opening extending at least partially through the housing, the opening in communication with the membrane and configured to be coupled to a vacuum source such that, when the vacuum source is coupled to the opening, fluid passing through the passageway is subjected to the vacuum generated by the vacuum source through the membrane.

2. The cassette of claim 1, wherein the membrane is configured to allow passage of gasses through the membrane and prevent passage of the fluid through the membrane.

3. The cassette of claim 2, wherein the membrane is hydrophobic.

4. The cassette of claim 2, wherein the membrane extends generally parallel to the fluid passageway.

5. The cassette of claim 4, further comprising at least one additional membrane positioned in fluid communication with the fluid passageway, the at least one additional membrane extending generally parallel to the fluid passageway.

6. The cassette of claim 2, wherein the membrane extends generally perpendicular to the fluid passageway.

7. The cassette of claim 6, further comprising at least one additional membrane positioned in fluid communication with the fluid passageway, the at least one additional membrane extending generally perpendicular to the fluid passageway.

8. The cassette of claim 6, further comprising at least one additional membrane positioned in fluid communication with the fluid passageway, the at least one additional membrane extending generally perpendicular to the fluid passageway.

9. The cassette of claim 1, wherein the fluid passageway is defined by an inlet, an outlet, and a lumen extending between the inlet and the outlet.

10. The cassette of claim 9, wherein the inlet is configured to receive the fluid from a fluid source and the outlet is configured to output the fluid to a surgical instrument after dissolved gasses have been extracted from the fluid through the membrane.

11. An ophthalmic surgical system, comprising:
a fluid source;
a vacuum source;
a fluid passageway in fluid communication with the fluid source such that a fluid from the fluid source passes through the fluid passageway;
a membrane positioned in communication with the fluid passageway such that the fluid passing through the fluid passageway contacts the membrane; and
a vacuum passageway in communication with the vacuum source and the membrane such that a vacuum generated by the vacuum source is applied through the membrane to the fluid passing through the fluid passageway.

12. The system of claim 11, wherein the membrane is configured to allow passage of gasses through the membrane and prevent passage of the fluid through the membrane.

13. The system of claim 12, wherein the membrane is hydrophobic.

14. The system of claim 12, wherein the membrane extends parallel to the fluid passageway.

15. The system of claim 12, wherein the membrane extends perpendicular to the fluid passageway.

16. The system of claim 12, wherein the membrane extends at an oblique angle to the fluid passageway.

17. The system of claim 12, wherein at least portions of each of the fluid passageway, the membrane, and the vacuum passageway are positioned within a fluidics cassette.

18. An ophthalmic surgical method, comprising:
introducing a fluid into a fluid passageway, wherein a membrane is positioned in communication with the fluid passageway such that the fluid passing through the fluid passageway contacts the membrane;
applying a vacuum to the fluid through the membrane such that dissolved gasses within the fluid are removed through the membrane; and
introducing the fluid into the eye after removal of the dissolved gasses through the membrane.

19. The method of claim 18, wherein the fluid is an electrolyte solution.

20. The method of claim 19, wherein the membrane is hydrophobic such that the fluid does not pass through the membrane.
